(19) United States
(12) Patent Application Publication
(10) Pub. No.: US 2011/0017206 A1
(43) Pub. Date: Jan. 27, 2011

(54) APPARATUS AND METHODS FOR DIRECT INHALATION OF SCENTS AND MEDICINES

(76) Inventor: Greg Fisher, Boston, MA (US)

Correspondence Address: Greg Fisher, 463 Shawmut Ave, Boston, MA, 02118, US

(21) Appl. No.: 12/841,487

(22) Filed: Jul. 22, 2010

Related U.S. Application Data
(60) Provisional application No. 61/227,890, filed on Jul. 23, 2009.

(51) Int. Cl. A61M 15/08

(52) U.S. Cl. 128/200.24

(57) ABSTRACT

An intranasal tab may provide temporary, controlled and direct inhalation of scents and/or medicine into the nose, thereby increasing the overall effectiveness and velocity of impact that scents and/or medicine may have on the mind and/or body. The tabs may provide increased convenience, speed, comfort and effectiveness of mobile aromatherapy and medicinal therapy. The tabs may be substantially flat, triangular, and designed from various types of absorbent materials. The center of the tab may be comprised of highly absorbent material compared to the perimeter and external surfaces of the tab which remain relatively dry to the touch. The tabs are adapted to be inserted separately into either nostril of the nose without falling out.

### CROSS-REFERENCE TO RELATED APPLICATIONS

This application claims the benefit of priority of U.S. Provisional patent application No. 61/227,890, filed Jul. 23, 2009, herein incorporated by reference.

### BACKGROUND OF THE INVENTION

The present invention relates to intranasal delivery apparatus and methods and, more particularly, to apparatus and methods for the direct inhalation of scents and/or medicines into a person's airway.

The human nose is an under-optimized interface between the human body and external environment. The human nose can distinguish between hundreds of substances, even in minute quantities, while the human tongue, on the other hand, can only distinguish among five qualities of taste. The sense of smell is the only one of the traditional five senses without a commonly utilized and mobile device that closely and directly interacts with that sense. The other four senses have widely available devices or mechanisms to enhance, complement or manipulate each of those senses—glasses for sight, oven mitt for touch, spices for taste, and hearing aids for hearing are just some examples.

Aromatherapy is a form of alternative treatment to influence mood, cognitive function and/or health through one's sense of smell. While specific efficacy of aromatherapy remains unproven, some preliminary clinical studies show positive results and other studies are currently underway. Currently available aromatherapy methods lack undetectable and mobile devices that enable the delivery of scents intranasally.

Pharmaceutical companies are testing ways to deliver vaporized or atomized drugs through the nose. Currently available medicinal nasal delivery methods may provide a burst of drug substance inside the nose but lack an apparatus designed to release medicine into one's airway over a period of time.

Existing intranasal delivery devices may be two-piece devices held together by a connector. The connector must always be somewhat visible since it connects externally below the septum. Visibility while in use may be an obstacle to broad acceptance in the marketplace. Existing devices may not optimize air flow into and out of the nose. Instead, they may fill a significant portion of the nasal space and restrict air flow, which is counterproductive, unless the device is intended to be used as a nasal filter. Existing devices may also lack the functional design to contain sufficient amounts of scents and/or medicine to be most effective.

As can be seen, there is a need for an effective and undetectable apparatus and method to deliver scents and/or drugs directly into one's nasal passage over a period of time.

### SUMMARY OF THE INVENTION

In one aspect of the present invention, a nasal delivery device comprises a triangularly shaped tab designed from certain materials to allow scents or medicines infused into the tab to evaporate into one's airway.

In another aspect of the present invention, the internal core of the tab is made from highly absorbent material to maximize the amount of scents or medicine absorbed into the center of the tab as compared to the external layer of the tab which is designed to remain dry to the touch.

In another aspect of the present invention, the triangular tab is substantially flat to allow air to flow over both sides of the tab when inserted into the nose.

These and other features, aspects and advantages of the present invention will become better understood with reference to the following drawings, description and claims.

### BRIEF DESCRIPTION OF THE DRAWINGS

FIG. 1 is a top view of an intranasal tab according to an embodiment of the present invention;

FIG. 2 is a side view of the intranasal tab of FIG. 1;

FIG. 3 is a kit designed to contain a number of the intranasal tabs of FIG. 1;

FIG. 4 shows a side-view of the tab of FIG. 1 inserted into a user's nostril; and

FIG. 5 shows a view of the inserted tab in a user's nostril from below.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, an embodiment of the present invention provides for direct inhalation of aromas and/or medicines into the nose, thereby affecting the impact scents and/or medicines may have on the mind and body. Embodiments of the present invention may provide increased mobility, convenience, speed, comfort and effectiveness of aromatherapy and medicinal therapy.

The apparatus of the present invention may be a substantially flat, triangular tab that may be made from a combination of highly absorbent and lesser absorbent materials. The center of the tab may contain highly absorbent material to optimally absorb scents and/or medicines thereinto, leaving the external surfaces of the tab relatively dry as compared to the more dense inner portion of the tab.

The tab of the present invention may be less complex and better designed than conventional intranasal delivery devices. Embodiments of the present invention may include single, contiguous tabs that may be inserted separately into one or both nostrils. Since there is no connector to join two tabs, as is included in conventional intranasal devices, the tabs of the present invention may be entirely invisible once inserted. The substantially flat design may also provide greater capacity to contain scents than conventional devices and allow for better air flow into and out of the nose. The external surfaces of the tab are relatively dry to touch which reduces the extent to which scents and/or medicines come into direct contact with the skin and, as a result, decreases the potential for irritation of the nose. The triangular tab may also include rounded corners and pliable perimeter edges, allowing the tab to fit comfortably inside the nose and prevent the tab from being inserted too far or falling out of the nose while in use.

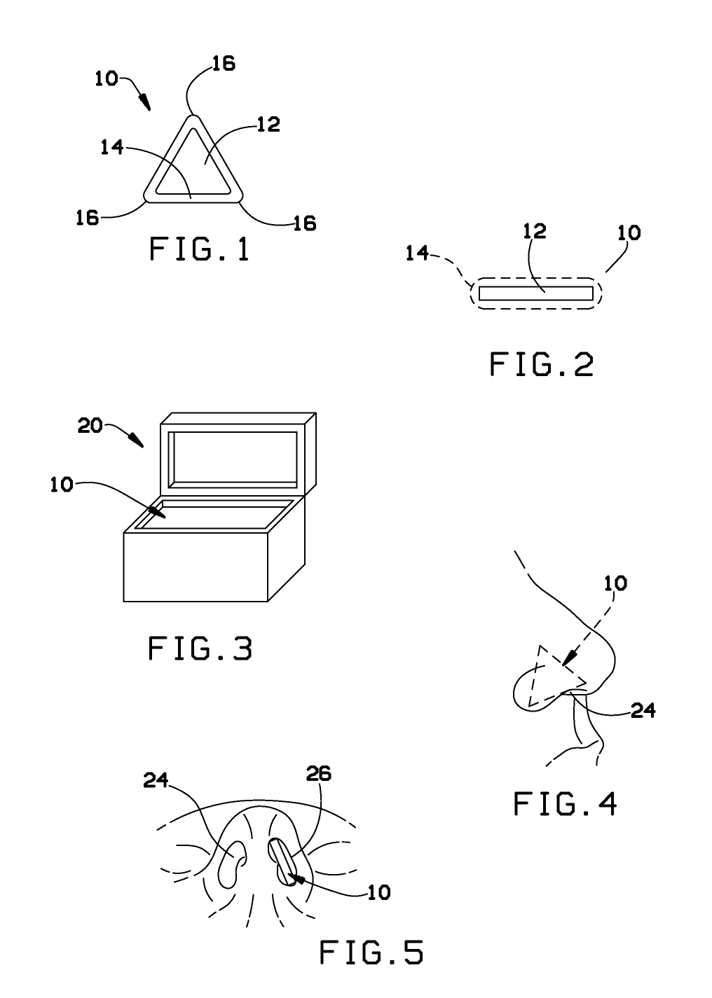

Referring to FIGS. 1, 2, and 4 an intranasal tab 10 has an equilateral triangular shape so that any of the three corners may be inserted up into the nose. Each side of the tab 10 may be from about 0.5 to about 1.0 inch long, typically about 0.75 inch. The tab 10 may be from about 0.05 to about 0.15 inches thick, typically about 0.10 inches thick. The tab 10 may be formed from a combination of absorbent materials.

The tab 10 may include a dense core 12 surrounded by a less dense and/or softer perimeter edge 14. The perimeter edge 14 is typically about 0.10 to 0.15 inches wide. The core 12 and perimeter 14 may be formed from two different types of material, with the core 12 being made from highly absorbent material, such as cotton yarn, high filament nylon or polyester, and with the perimeter 14 being made from a softer, lesser absorbent material, such as a monofilament polyester or polypropylene. The perimeter edge 14 is better able to be molded to fit the contours of one's nasal cavity than the more dense core 12.

The same material used in the perimeter 14 may also be present covering the entire exterior surface 17 of the tab 10 resulting in a soft outer layer for additional comfort. The lesser absorbent external layer 17 also allows scents and/or medicines to concentrate in the center of the tab within the higher absorbent material leaving the external layer of the tab relatively dry to the touch. When inserted into the nose, the relatively dry exterior surface 17 (as compared to the core 12) may avoid irritation to the nasal cavity by minimizing the scented oil/fragrance or medicine from coming into direct contact with the mucus membranes of the inner nose. Air is able to pass through the dense core 12 as well as the external layer 17 of the tab to aid in breathability.

As discussed above, the shape of the tab 10 may be flat and triangular. The tab 10 may have rounded corners 16 which may allow a natural and comfortable fit inside the nose. The outside perimeter edge 14 of the tab 10 may be pliable so the tab 10 can easily mold into the nostril. Referring to FIG. 5, the tabs 10 may be designed to fit snugly into the lower part of the nose so that when a tab is inserted into the nose, the opposite edge of the corner inserted into the nose may be flush with the opening of the nose. Once inserted, the tab 10 may not be visible in the nose (unless looking directly up into the nasal cavity). The slim design of the tab allows the tab to be placed in the center of the nostril so air can pass over both sides of the tab. This design allows scents/medicines to be dispersed from both sides of the tab while also improving breathability.

Essential oils, fragrances or medicines may be infused into the tab. Natural and/or artificial scents may be used. The scents, fragrances and/or medicines may be gradually released over a period of 30 minutes to 1 hour. The specific dose amounts of each scent and/or medicine may depend on the individual scent and/or medicine, as well as how the tabs are marketed—for example, as regular strength or extra strength.

Referring to FIG. 3, a kit 20 may include individually wrapped tabs 10 in a container. The kit 20 may include, for example twelve tabs 10, individually wrapped in cellophane, for example, to maintain freshness. The kit 20 may contain tabs of a single fragrance or multiple fragrances, for example. A purchaser may be able to select a certain scent based on the mood sought to be achieved at any particular time. For example, scents may include rose, cedar, lime, chocolate, coffee, sandalwood, sage and lavender. Tabs may be selected for mood, for energy, to complement food and beverages, or the like. In one embodiment, the kit 20 may include pre-infused tabs. In an alternate embodiment, the kit 20 may include dry, un-infused tabs with a liquid to be infused into the tab when ready for use. The liquid may be an essential oil, an artificial fragrance, a medicine, or the like.

Referring to FIG. 4, a user 22 may insert the tab 10 into a first nostril 24. In addition, referring to FIG. 5, the user 22 may optionally insert a second tab 10 into a second nostril 26. Once inserted, the tabs 10 may not be visible from an ordinary view of the user 22. When the user 22 tips their head back, one may view the tabs 10 inserted into each nostril 24, 26.

It should be understood, of course, that the foregoing relates to exemplary embodiments of the invention and that modifications may be made without departing from the spirit and scope of the invention as set forth in the following claims.

What is claimed is:

1. An intranasal delivery device comprising:
an equilaterally-shaped triangular tab formed of materials adapted to disperse scents and/or medicines; and
a core of the tab having a highly absorbent material relative to an exterior layer of the tab.

2. The device of claim 1, wherein the tab is substantially flat with rounded corners.

3. The device of claim 2, with a pliable perimeter edge adapted to be inserted separately into a single nostril by conforming to one's inner nose.

4. The device of claim 3, wherein:
the scent, fragrance or medicine is pre-infused into the tab;
a plurality of tabs are individually wrapped; and the plurality of wrapped tabs is inserted into a kit.
